(19) United States
(12) Patent Application Publication
(10) Pub. No.: US 2025/0054645 A1
(43) Pub. Date: Feb. 13, 2025

(54) AUTOMATED ORDERS AND RESULT REPORTING FOR PRESCRIPTION-BASED USER-ADMINISTERED DIAGNOSTIC TESTING

(71) Applicant: Becton, Dickinson and Company, Franklin Lakes, NJ (US)

(72) Inventors: Rajeev Sehgal, Cockeysville, MD (US); Susan de la Torre, San Diego, CA (US); Sandeep Mukund Naik, Bridgewater, NJ (US); Neeraj Kapoor, Anaheim, CA (US)

(21) Appl. No.: 18/928,953

(22) Filed: Oct. 28, 2024

Related U.S. Application Data
(63) Continuation of application No. PCT/US2023/067139, filed on May 17, 2023.
(60) Provisional application No. 63/365,104, filed on May 20, 2022.

(51) Int. Cl. G16H 80/00; G16H 10/40

(52) U.S. Cl. CPC G16H 80/00; G16H 10/40

(57) ABSTRACT

Systems and methods for integration of user-administered diagnostic test results with electronic health records are provided. In one aspect, a method includes receiving a prescription specifying a user-administered test and a patient, transmitting prescription information to a pharmacy and to the test provider, generating a unique order identifier which is sent to a user application that associates the order identifier with a test result after the user administers the test, receiving the test result in association with the order identifier, storing the test result and the order identifier in a medical record database in association with a medical record number or a consultation identifier, and sending a notification of the test result to the prescribing clinician.

### CROSS-REFERENCE TO RELATED APPLICATIONS

This application is a continuation of International Application No. PCT/US2023/067139, filed May 17, 2023, which claims the benefit of U.S. Provisional Application No. 63/365,104, filed May 20, 2022, both of which are hereby incorporated by reference in their entirety.

### TECHNICAL FIELD

The present disclosure relates in general to diagnostic testing, and more particularly to systems and methods for integration of user-administered diagnostic test results with electronic health records.

### BACKGROUND

User-administered diagnostic tests are becoming increasingly common as they can provide reliable, inexpensive, portable, rapid, and simple diagnostic tests. Advantageously, many user-administered diagnostic tests can be minimally invasive and can be performed by a user (e.g., a patient or other non-clinician) in a setting outside of a clinical or lab environment, such as a user's home. However, sharing results of user-administered diagnostic tests is typically difficult, time-consuming, and inefficient in conjunction with existing electronic health record systems.

### SUMMARY

In a first aspect, a system includes one or more processors associated with at least one of a healthcare provider and a testing provider, and a user device comprising one or more processors. The one or more processors associated with at least one of the healthcare provider and the testing provider are configured with computer-executable instructions to: receive, from a clinician computing device, a prescription specifying a user-administered test and a patient; transmit, to a pharmacy interface, prescription information corresponding to the prescription; generate an order identifier uniquely identifying the prescription; and cause transmission of the order identifier to a user application installed on a computing device associated with the patient. The one or more processors of the user device are configured with processor-executable instructions included in the user application to: receive the order identifier; obtain a test result corresponding to the user-administered test; associate the received order identifier with the test result; and send the test result to the one or more processors associated with the healthcare provider in association with the order identifier.

In some embodiments, the one or more processors associated with at least one of the healthcare provider and the testing provider are further configured to: receive the test result in association with the order identifier; and store the test result and the order identifier in a medical record database in association with at least one additional identifier corresponding to the patient or to a consultation corresponding to the prescription.

In some embodiments, the one or more processors associated with at least one of the healthcare provider and the testing provider are further configured to send, to the clinician computing device, a notification indicative of the test result.

In some embodiments, the one or more processors associated with at least one of the healthcare provider and the testing provider are further configured to cause transmission of a notification instructing the patient to install the user application on the computing device. In some embodiments, the notification includes an email, a text message, or a push notification in a second application different from the user application.

In some embodiments, obtaining the test result corresponding to the user-administered test includes: receiving an image of a test device taken following application of a specimen to the test device; and automatically determining the test result based on image data of the received image of the test device. In some embodiments, the test device includes a lateral flow assay test strip, and automatically determining the test result includes detecting one or more lines on the lateral flow assay test strip.

In some embodiments, the one or more processors associated with at least one of the healthcare provider and the testing provider are further configured to cause transmission to the user application of an identifier of a test type corresponding to the user-administered test. In some embodiments, the transmission of the identifier of the test type causes the user application to provide, to the patient, one or more instructions associated with the test type.

In some embodiments, the user-administered test includes a lateral flow assay and the test result includes one or more lines on the lateral flow assay.

In a second aspect, a computer-implemented method for provision of a user-administered test in conjunction with an electronic medical record system includes, under control of one or more processors: receiving, from a clinician computing device, a prescription specifying a user-administered test and a patient; transmitting, to a pharmacy interface, prescription information corresponding to the prescription; generating an order identifier uniquely identifying the prescription; causing transmission of the order identifier to a user application installed on a computing device associated with the patient and configured to associate the order identifier with a test result determined at least in part by the user application; receiving, from the user application, the test result in association with the order identifier; storing the test result and the order identifier in a medical record database in association with at least one additional identifier corresponding to the patient or to a consultation corresponding to the prescription; and sending, to the clinician computing device, a notification associated with the test result.

In some embodiments, the method further includes, prior to causing transmission of the order identifier to the user application, sending a notification instructing the patient to install the user application on the computing device. In some embodiments, the notification instructing the patient to install the user application includes an email, a text message, or a push notification in a second application different from the user application.

In some embodiments, the method further includes causing transmission to the user application of an identifier of a test type corresponding to the user-administered test. In some embodiments, the transmission of the identifier of the test type causes the user application to provide, to the patient, one or more instructions associated with the test type. In some embodiments, the identifier of the test type corresponds to a lateral flow assay test.

In some embodiments, the notification associated with the test result further includes an identifier of the patient or of a consultation associated with the prescription.

In some embodiments, the user-administered test includes a lateral flow assay test, and the test result includes one or more lines on the lateral flow assay.

In a third aspect, a computer-implemented method for provision of a user-administered test in conjunction with an electronic medical record system includes, under control of one or more processors of a computing device associated with a patient: receiving, from a computing system associated with a testing provider or a healthcare provider, an order identifier corresponding to a prescription specifying a user-administered test and the patient; obtaining a test result corresponding to the user-administered test; and sending, to the computing system associated with the testing provider or the healthcare provider, the test result in association with the order identifier.

In some embodiments, the method further includes: receiving an indicator of a test type of the user-administered test; obtaining one or more instructions associated with the test type; and displaying the one or more instructions to the patient prior to obtaining the test result. In some embodiments, the one or more instructions include instructions for collecting a sample, instructions for application of a sample to a test device, instructions for determination of adequacy of a sample or a test result, or instructions for imaging a test device for determination of a result by the computing device.

In some embodiments, obtaining the test result corresponding to the user-administered test includes prompting the patient to input the test result at a user interface of the computing device.

In some embodiments, obtaining the test result corresponding to the user-administered test includes: receiving an image of a test device used for the user-administered test; and automatically determining the test result based on image data of the received image of the test device. In some embodiments, the test device includes a lateral flow assay test strip, and automatically determining the test result includes detecting one or more lines on the lateral flow assay test strip. In some embodiments, the computing device includes an image capture device, and receiving the image of the test device includes: displaying, to the user, an instruction to capture an image of the test device using the image capture device; and receiving the image from the image capture device.

In some embodiments, the method further includes providing a notification to the patient in response to receiving the order identifier.

In some embodiments, the user-administered test includes a lateral flow assay and the test result includes one or more lines on the lateral flow assay.

### BRIEF DESCRIPTION OF THE DRAWINGS

FIG. 1 schematically illustrates an example system and closed-loop communication workflow for implementing user-administered diagnostic testing in conjunction with an electronic health record system.

FIG. 2 is a flow chart illustrating an example method for obtaining and reporting a result of a user-administered diagnostic test based on a test order from a healthcare provider.

### DETAILED DESCRIPTION

Conventionally, tests ordered, requested, or prescribed by physicians or other clinicians have been performed in settings such as hospital labs or reference labs which may employ trained test administration personnel and computer-implemented workflows that allow for receiving test prescriptions and transmitting test results that can be entered into a patient record. However, the development of high-quality rapid diagnostic tests has enabled the deployment of laboratory quality diagnostic assays into environments which historically were unable to process samples. For example, an increasing number of tests which were traditionally performed in a laboratory setting are now suitable for user-administration, such as by a patient in their own home. Advantageously, such user-administered tests may be easier and more cost-effective than laboratory-administered tests. In the context of testing for the presence of an infectious disease, user-administered tests can also be performed without requiring a possibly sick patient to travel to a laboratory or clinical setting where pathogens may be spread. In some cases, user-administered tests may be desirable for tests that are ordered following a virtual consultation with a physician or other clinician, as a hospital or laboratory test may require an additional trip to a healthcare provider facility.

Existing medical record systems do not provide for efficient integration of the results of user-administered tests. For example, if a patient administers an at-home diagnostic test and wishes to share the results with their physician, they may need to manually send the test results via email or fax, or may need to log into a medical record tracking application and manually log the result within the application. Such processes may be time-consuming and error-prone, and rely on several manual communication steps in order to have results entered into a patient's medical record. In addition, because user-administered tests are frequently purchased over the counter, it may be difficult for users to be reimbursed for the costs of such tests that would otherwise be covered by insurance.

The present disclosure provides systems and workflows that can address these challenges, for example, by allowing user-administered diagnostic tests to be integrated within prescription or laboratory test reporting workflows, as will be described in greater detail. Advantageously, the systems, workflows, and methods of the present disclosure can reduce or minimize disruption to existing workflows employed by physicians in prescribing or ordering medications and tests, as they allow a physician to order or prescribe a user-administered test in substantially the same manner they would otherwise order a laboratory or hospital test, and to receive an automatic notification of the results of the user-administered test in the same manner that would otherwise be received from existing laboratory or hospital tests.

Embodiments of the present disclosure relate to systems and techniques for detection of analytes of interest that may be present in biological or non-biological samples such as fluids. Analytes of interest may include any detectable substances such as but not limited to antibodies, proteins, haptens, nucleic acids, amplicons, and hormones. Embodiments of the present disclosure are described with reference to diagnostic tests, but it will be understood that the systems, workflows, and methods of the present disclosure can be implemented in user-administered non-diagnostic testing, for example testing for hazardous or non-hazardous drugs or contaminants. Throughout this disclosure, example systems, devices, and methods will be described with reference to collection, testing, and detection of analytes such as those relevant for diagnostic testing related to infectious diseases, but it will be understood that the present disclosure can be used to collect, test, and detect any particle, molecule, or analyte of interest. Test devices or systems such as user-administered tests as described herein may be configured for performance of diagnostic and/or non-diagnostic tests. In some embodiments, embodiments of the present disclosure can be implemented in conjunction with systems, such as the BD Veritor At-Home COVID-19 Test, the BD Veritor System for Rapid Detection of SARS COV-2, the BD Veritor System for Rapid Detection of Flu A+B, the BD Veritor System for Rapid Detection of SARS COV-2 & Flu A+B, the BD Veritor System for Rapid Detection of Respiratory Syncytial Virus (RSV), the BD Veritor System for Rapid Detection of Group A Strep, the BD Veritor system, the BD Veritor Plus system, and/or components or operations thereof.

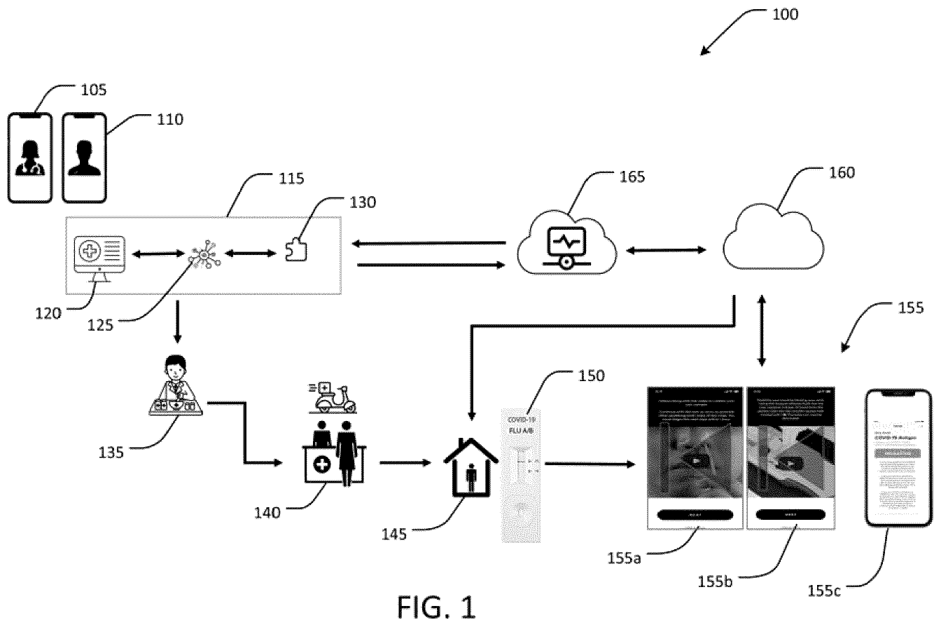

FIG. 1 schematically illustrates an example system 100 and closed-loop communication workflow for implementing user-administered diagnostic testing in conjunction with an electronic health record system. The system 100 is exemplary and includes a number of optional components; it will be understood that the workflows and methods of the present disclosure may equally be implemented in other systems having more, fewer, or different components relative to the system 100 of FIG. 1.

The example system 100 can include a clinician computing device 105, a patient computing device 110, and a healthcare provider (HCP) computing system 115, which may be in communication with one or more networked resources such as a testing provider application 160 and/or a middleware application 165. The HCP computing system 115 can include components such as an electronic medical record (EMR) database 120, a middleware interface component 130, and an interface engine 125. The HCP computing system 115 can be configured to communicate with a pharmacy interface 135 associated with at least one pharmacy 140. The system 100 can further include a testing provider user application 155 configured to communicate with the testing provider application 160.

The clinician computing device 105 and the patient computing device 110 can be, for example, mobile devices such as smartphones, tablets, or the like, laptop or desktop computing systems, or any other computing devices. The clinician computing device 105 may include functionality for audio or video conferencing, such as for conducting a virtual consultation with a patient. The clinician computing device 105 may further be configured to communicate with the HCP computing system 115 via a network, such as to allow a clinician to order or submit prescriptions for a patient following a virtual or in-person consultation. The patient computing device 110 may similarly include functionality for audio or video conferencing. The patient computing device 110 may be configured to communicate with the clinician computing device 105 via a network, either directly or through one or more components of the HCP computing system 115, such as to enable the patient to participate in a virtual consultation with a clinician using a clinician computing device 105.

The HCP computing system 115 can be in communication with any or all of the clinician computing device 105, the patient computing device 110, the pharmacy interface 135, the testing provider application 160, and/or the middleware application 165. Moreover the HCP computing system 115 can be in communication with a plurality of different clinician computing devices 105, patient computing devices 110, pharmacy interfaces 135, testing provider applications 160, and/or middleware applications 165. The EMR database 120 may store electronic medical records for a number of patients of the HCP. Individual patient medical records may include various information such as records of clinician visits and/or consultations, presently or previously prescribed treatments, medications, tests, and the like, and any other information commonly stored in association with electronic medical records or electronic health records. The middleware interface component 130 is configured for communication between the middleware application 165 and the HCP computing system 115. The interface engine 125 can be configured for communication between the EMR database 120, the middleware interface component 130, and one or more pharmacy interfaces 135. Some embodiments of the systems, workflows, and methods of the present disclosure can be implemented without the middleware application 165 and/or the middleware interface component 130.

The pharmacy interface 135 can be any computing system configured for interaction between healthcare providers and pharmacies. For example, the pharmacy interface 135 can be a computing system of an e-prescription provider or any other health information exchange provider. The pharmacy interface 135 can be in communication with one or a plurality of pharmacies 140, which may be physical pharmacy locations at which patients or other users can pick up prescribed medications, tests, and the like, and/or delivery services which can provide prescribed medications, tests, and the like to patients or other users.

The testing provider user application 155 can be a software program such as a mobile device application, which can be executed on a device such as a patient computing device 110 or another computing device operable by a user, such as a patient or another user assisting a patient in taking a diagnostic test. The testing provider user application 155 can perform any number of functions, including but not limited to providing instructions to a user for administering a test (e.g., instructions for collection of a sample, instructions for application of the sample to a testing device, instructions for determination of adequacy of a sample and/or a test result, instructions and/or an interface for imaging a test device for determination of a result by the testing provider user application 155, etc.), receiving an input test result from a user, optically determining a result of a test result based on one or more images of a test device, associating a test result with one or more identifiers, and/or transmitting a test result and any associated identifiers via a network to a remote computing system such as a testing provider application 160.

An example closed-loop communication workflow for implementing user-administered diagnostic testing will now be described with reference to the components of the system 100. It will be understood that the following workflow may be implemented in conjunction with other systems having more, fewer, or different components than those illustrated in FIG. 1 without departing from the scope of the present disclosure.

In a first step, a patient has a consultation with a clinician or other healthcare provider, for example, a physician. The consultation may be a virtual consultation in some embodiments, in which the patient may use the patient computing device 105 to communicate with a clinician using the clinician computing device 110. As a result of the consultation, the clinician may decide to order a user-administered test. For example, if the clinician identifies symptoms indicative of a possible infection with an infectious disease, the clinician may order a user-administered test to determine whether the patient has the infectious disease.

During or after the consultation, the clinician may enter a prescription or order for the user-administered test for the patient using a prescription interface on the clinician computing device 105 or another computing device. The prescription or order is entered into a record for the patient in the EMR database 120. The prescription or order can further be associated with a consultation identifier (e.g., a visit identifier or visit ID) uniquely identifying the consultation or visit at which the user-administered test was prescribed. When the prescription or order has been entered into the patient's record, the interface engine 125 can generate a pharmacy order which can be sent to the pharmacy interface 135 to be fulfilled at a pharmacy. In addition, the interface engine 125 can duplicate the order and send the duplicated order to the middleware application 165 via the middleware interface component 130. The order sent to the middleware application 165 can include one or more associated identifiers, such as a generated order identifier associated with the requested test, a medical record number, and/or the consultation identifier. The middleware application 165 can send the order and any associated identifiers on to the testing provider application 160.

In response to receiving the order and associated identifiers, the testing provider application 160 can send a communication to the patient. For example, the testing provider application 160 can send a notification to the testing provider user application 155 executing on the patient computing device 110 or another computing device, which can indicate the type of user-administered test to be administered in conjunction with the application. The notification can further include the received order identifier, medical record number, and/or consultation identifier so that any such identifier can later be associated with a test result. In some embodiments, the testing provider application 160 can further be configured to send a notification to the patient (e.g., an email, a text message, a push notification in an application such as a medical record access application, etc.) to download the testing provider user application 155 if the patient or an associated user has not yet downloaded the testing provider user application 155.

The pharmacy interface 135, in response to receiving the pharmacy order from the HCP computing system 115, can transmit the pharmacy order to a physical pharmacy 140, such as a pharmacy selected by the patient. The patient or an authorized user can then pick up the prescribed user-administered test 150 from the pharmacy 140 and can transport the user-administered test 150 to a testing location 145, such as the patient's home or another suitable location for taking the test.

At the testing location 145, the patient or another user administers the user-administered test 150. The user-administered test 150 may be administered in conjunction with the testing provider user application 155. For example, at 155a, a graphical user interface (GUI) of the testing provider user application 155 may provide one or more instructions for collecting a sample from the patient, such as by displaying one or more written instructions, photos, diagrams, videos, animations, or the like. At 155b, the GUI may further provide one or more instructions for applying the sample to the test, such as by applying the test to a lateral flow assay or other test analysis device. At 155c, the GUI may further provide a visual output locally indicating a result of the test to the patient and/or other user administering the test. In some embodiments, the result of the test may be determined partially or entirely by the testing provider user application 155.

The testing provider user application 155 can further associate the test result with identifying information corresponding to the consultation, patient, and/or test. For example, the testing provider user application 155 may have stored information such as the order identifier, medical record number, and/or consultation identifier. The testing provider user application 155 can retrieve the stored information and transmit the test result, in association with one or more of the stored identifiers, to the testing provider application 160. For example, the testing provider user application 155 can transmit a communication to the testing provider application 160 that includes the test result and at least one of the order identifier, the consultation identifier, and/or the medical record number. The testing provider application 160 can then transmit the test result and any associated identifiers to the HCP computing system 115, either directly or via the middleware application 165.

The HCP computing system 115 receives the test result and associated identifiers from the testing provider application 160. Upon receiving the communication, the HCP computing system 115 can process the communication to associate the test result with the patient's EMR and with the specific consultation in the EMR database 120. In some embodiments, the HCP computing system 115 uses an existing processing workflow for unsolicited laboratory testing results to process the test result and to generate an order record which can be associated in the EMR database 120 with the patient and the consultation corresponding to the user-administered test 150. In accordance with an unsolicited test result workflow or any other suitable test result receipt workflow, the HCP computing system 115 can update the patient's EMR based on the test result and can further send a notification of the test result to the patient's healthcare provider and/or to the individual clinician that performed the consultation. Accordingly, the workflow described above can efficiently allow a clinician to prescribe a user-administered test and be automatically notified of the test result in a manner that reduces the probability of errors, missing test results, or other inefficiencies.

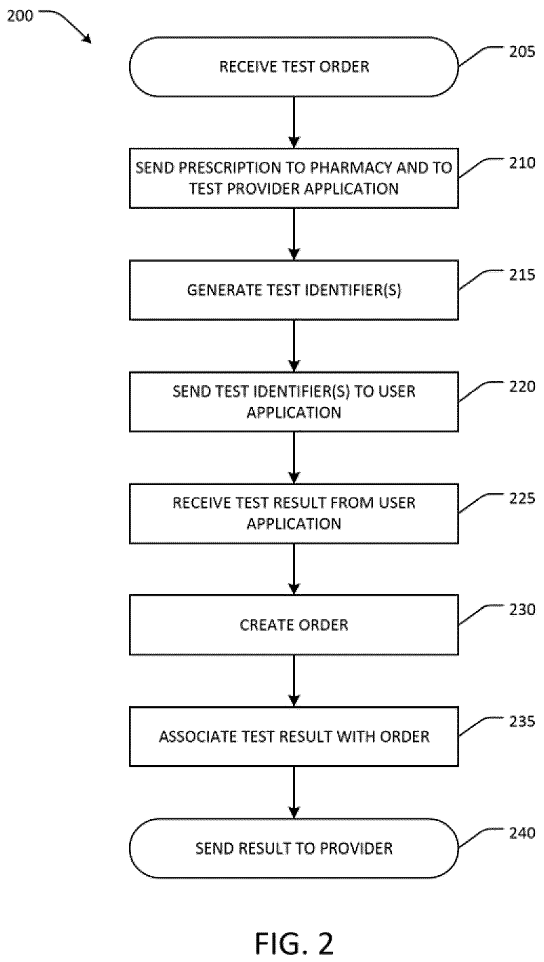

FIG. 2 is a flow chart illustrating an example method 200 for obtaining and reporting a result of a user-administered diagnostic test based on a test order from a healthcare provider. The method 200 can be performed by a computing system such as, for example, the HCP computing system 115 and the testing provider application 160 in the system 100 of FIG. 1. Although the method 200 will be described with reference to the HCP computing system 115 and other components of the system 100, the method 200 can equally be performed in conjunction with other computing systems without departing from the scope of the present disclosure.

The method 200 begins at block 205 when a test order is received. The test order may be received, for example, as a result of a physician or other clinician entering a prescription for a specified user-administered test using a computer prescription interface. The test order received at block 205 may be associated with an identifier of a specific type of user-administered test, a medical record number associated with a patient who is to be tested, and/or a consultation identifier identifying the visit or consultation at which the user-administered test was prescribed. When the test order has been received, the method 200 continues to block 210.

At block 210, the HCP computing system 115 sends the prescription to a pharmacy and to a test provider application. The prescription can be sent to the pharmacy, for example, via a pharmacy interface such as the pharmacy interface 135 of FIG. 1. Upon receiving the prescription, the pharmacy may retrieve the specified user-administered test and provide the test to the patient.

At block 215, the HCP computing system 115 and/or the testing provider application 160 can generate one or more test identifiers such as, for example, an order identifier. For example, the one or more test identifiers can include an identifier to be associated with a subsequently created order record corresponding to the test and its result once the test is taken. The one or more test identifiers can subsequently be used as a unique identifier of the result of the test.

At block 220, the HCP computing system 115 and/or the testing provider application 160 can send the one or more test identifiers to a user application (c.g., testing provider user application 155) associated with the patient. In some embodiments, if the patient or an associated user has not yet downloaded the user application, a notification directing the patient or user to download the application may be sent to the patient or other user via a different means of communication such as email, text, or via another application.

At block 225, after the patient has self-administered the user-administered test or another user has administered the user-administered test to the patient in conjunction with the user application, the HCP computing system 115 receives a test result from the user application. For example, the HCP computing system 115 may receive the test result via the testing provider application 160 and/or the middleware application 165. The received test result communication can include the one or more test identifiers generated at block 215, such that the HCP computing system can accurately associate the received test result with the patient's electronic medical record or electronic health record in the EMR database 120.

At block 230, in response to receiving the test result communication directly or indirectly from the user application, the HCP computing system 115 creates an order using an order creation workflow. For example, in some embodiments, the order can be created for the patient using an existing unsolicited test result workflow. The order can be associated with a medical record number corresponding to the patient and/or with a consultation identifier corresponding to the particular visit or consultation at which the test was prescribed.

At block 235, when the order has been created, the HCP computing system associates the received test result with the order. For example, the order record can be augmented to include an indication of whether the patient was determined to have a positive or negative test result for the presence of a particular analyte or analytes of interest, which may be indicative of whether the patient has or does not have a specified infectious disease or other condition detectable by the specified user-administered test. After the test result is associated with the order, the method 200 terminates at block 240 as the test result is sent to the patient's healthcare provider. The test result may be provided, for example, as a notification to the clinician who conducted the initial consultation or visit and prescribed the test, such that the clinician is alerted to the test result without having to follow up with the patient, rely on a patient's manual reporting, or manually check the patient's electronic medical record to retrieve the result of the test and take any indicated actions, such as prescribing a medication or other course of treatment associated with the test result.

### Terminology

Analytical devices described herein may include diagnostic tests or any other testing devices, assays, and/or systems for detecting one or more analytes of interest in a sample. In the context of the present disclosure, user-administered tests may include a variety of tests configured to be administered to a patient or other individual to be tested, by a user who is not necessarily a physician, another clinician, a laboratory technician, or the like. For example, the user administering a user-administered test may be the patient or individual being tested, or another person such as a family member, caregiver, acquaintance, or the like. User-administered tests can also be configured to be administered in a variety of locations other than a laboratory or clinical setting. A user-administered test may be an “at-home” test suitable for administration at a user's home, workplace, or any other public or private location. For example, a user-administered test may be a packaged kit including all equipment necessary to collect and test a biological or environmental sample to generate a test result. Moreover, user-administered tests may be configured to be used in conjunction with an application executed on a computing device, such as a patient's or user's mobile device or any other computing device. In such computer-integrated tests, the application may be configured only to receive test results input by a user, or may be configured to perform at least a portion of the determination of a test result, for example, by taking or receiving one or more images of a test device such as an assay and optically generating a test result based on the one or more images.

Analytical devices described herein can accurately measure a plurality of analytes of interest in many different kinds of samples. Samples can include a specimen or culture obtained from any source, as well as biological and environmental samples. Biological samples may be obtained from animals (including humans) and encompass fluids, solids, tissues, and gases. Biological samples include urine, saliva, and blood products, such as plasma, serum and the like. Such examples are not however to be construed as limiting the sample types applicable to the present disclosure.

In some embodiments the sample is an environmental sample for detecting one or a plurality of analytes in the environment. In some embodiments, the sample is a biological sample from a subject. In some embodiments, a biological sample can include peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, or other lavage fluids.

As used herein, “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, and metabolites of or antibodies to any of the above substances. Specific examples of some analytes include ferritin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); interferon-induced GTP-binding protein (also referred to as myxovirus (influenza virus) resistance 1, MX1, MxA, IFI-78K, IFI78, MX, MX dynamin like GTPase 1); procalcitonin (PCT); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Additional analytes may be included for purposes of biological or environmental substances of interest.

The present disclosure relates to diagnostic and/or environmental testing devices such as, for example, lateral flow assay devices, test systems, and methods to determine the presence and concentration of a plurality of analytes in a sample, including when one or more analytes of interest are present at high concentrations and one or more analytes of interest are present at low concentrations. As discussed above, as used herein, “analyte” generally refers to a substance to be detected, for example a protein. Examples of proteins that can be detected by the lateral flow assay devices, test systems, and methods described herein include, without limitation:

TRAIL: TNF-related apoptosis-inducing ligand (also known as Apo2L, Apo-2 ligand and CD253); representative RefSeq DNA sequences are NC_000003.12; NC_018914.2; and NT_005612.17 and representative RefSeq Protein sequence accession numbers are NP_001177871.1; NP_001177872.1; and NP_003801.1. The TRAIL protein belongs to the tumor necrosis factor (TNF) ligand family.

CRP: C-reactive protein; representative RefSeq DNA sequences are NC_000001.11; NT_004487.20; and NC_018912.2 and a representative RefSeq Protein sequence accession numbers is NP_000558.2.

IP-10: Chemokine (C-X-C motif) ligand 10; representative RefSeq DNA sequences are NC_000004.12; NC_018915.2; and NT_016354.20 and a RefSeq Protein sequence is NP_001556.2.

PCT: Procalcitonin is a peptide precursor of the hormone calcitonin. A representative RefSeq amino acid sequence of this protein is NP_000558.2. Representative RefSeq DNA sequences include NC_000001.11, NT_004487.20, and NC_018912.2.

MX1: Interferon-induced GTP-binding protein Mx1 (also known as interferon-induced protein p78, Interferon-regulated resistance GTP-binding protein, MxA).

Representative RefSeq amino acid sequences of this protein are NP_001138397.1; NM_001144925.2; NP_001171517.1; and NM_001178046.2.

Lateral flow assay devices, test systems, and methods according to the present disclosure can measure either the soluble and/or the membrane form of the TRAIL protein. In one embodiment, only the soluble form of TRAIL is measured.

Implementations disclosed herein provide systems, methods and apparatus for detection of the presence and/or quantity of hazardous drugs. One skilled in the art will recognize that these embodiments may be implemented in hardware or a combination of hardware and software and/or firmware.

The assay reading functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, c.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. A computing environment can include any type of computer system, including, but not limited to, a computer system based on a microprocessor, a mainframe computer, a digital signal processor, a portable computing device, a personal organizer, a device controller, and a computational engine within an appliance, to name a few.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like. The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. Thus, the present invention is not intended to be limited to the implementations shown herein but is to be accorded the widest scope consistent with the principles and novel features disclosed herein.

1. A system comprising:
one or more processors associated with at least one of a healthcare provider and a testing provider, the one or more processors configured with computer-executable instructions to:
receive, from a clinician computing device, a prescription specifying a user-administered test and a patient;
transmit, to a pharmacy interface, prescription information corresponding to the prescription;
generate an order identifier uniquely identifying the prescription; and
cause transmission of the order identifier to a user application installed on a computing device associated with the patient; and
a user device comprising one or more processors, the one or more processors configured with processor-executable instructions included in the user application to:
receive the order identifier;
obtain a test result corresponding to the user-administered test;
associate the received order identifier with the test result; and
send the test result to the one or more processors associated with the healthcare provider in association with the order identifier.

2. The system of claim 1, wherein the one or more processors associated with at least one of the healthcare provider and the testing provider are further configured to:
receive the test result in association with the order identifier; and
store the test result and the order identifier in a medical record database in association with at least one additional identifier corresponding to the patient or to a consultation corresponding to the prescription.

3. The system of claim 2, wherein the one or more processors associated with at least one of the healthcare provider and the testing provider are further configured to send, to the clinician computing device, a notification indicative of the test result.

4. The system of claim 1, wherein the one or more processors associated with at least one of the healthcare provider and the testing provider are further configured to cause transmission of a notification instructing the patient to install the user application on the computing device.

5. The system of claim 4, wherein the notification comprises an email, a text message, or a push notification in a second application different from the user application.

6. The system of claim 1, wherein obtaining the test result corresponding to the user-administered test comprises:
receiving an image of a test device taken following application of a specimen to the test device; and
automatically determining the test result based on image data of the received image of the test device.

7. The system of claim 6, wherein the test device comprises a lateral flow assay test strip, and wherein automatically determining the test result comprises detecting one or more lines on the lateral flow assay test strip.

8. The system of claim 1, wherein the one or more processors associated with at least one of the healthcare provider and the testing provider are further configured to cause transmission to the user application of an identifier of a test type corresponding to the user-administered test.

9. The system of claim 8, wherein the transmission of the identifier of the test type causes the user application to provide, to the patient, one or more instructions associated with the test type.

10. The system of claim 1, wherein the user-administered test comprises a lateral flow assay and the test result comprises one or more lines on the lateral flow assay.

11. A computer-implemented method for provision of a user-administered test in conjunction with an electronic medical record system, the method comprising, under control of one or more processors:
receiving, from a clinician computing device, a prescription specifying a user-administered test and a patient;
transmitting, to a pharmacy interface, prescription information corresponding to the prescription;
generating an order identifier uniquely identifying the prescription;
causing transmission of the order identifier to a user application installed on a computing device associated with the patient and configured to associate the order identifier with a test result determined at least in part by the user application;
receiving, from the user application, the test result in association with the order identifier;
storing the test result and the order identifier in a medical record database in association with at least one additional identifier corresponding to the patient or to a consultation corresponding to the prescription; and
sending, to the clinician computing device, a notification associated with the test result.

12. The computer-implemented method of claim 11, further comprising, prior to causing transmission of the order identifier to the user application, sending a notification instructing the patient to install the user application on the computing device.

13. The computer-implemented method of claim 12, wherein the notification instructing the patient to install the user application comprises an email, a text message, or a push notification in a second application different from the user application.

14. The computer-implemented method of claim 11, further comprising causing transmission to the user application of an identifier of a test type corresponding to the user-administered test.

15. The computer-implemented method of claim 14, wherein the transmission of the identifier of the test type causes the user application to provide, to the patient, one or more instructions associated with the test type.

16. The computer-implemented method of claim 14, wherein the identifier of the test type corresponds to a lateral flow assay test.

17. The computer-implemented method of claim 11, wherein the notification associated with the test result further comprises an identifier of the patient or of a consultation associated with the prescription.

18. The computer-implemented method of claim 11, wherein the user-administered test comprises a lateral flow assay test, and wherein the test result comprises one or more lines on the lateral flow assay.

19. A computer-implemented method for provision of a user-administered test in conjunction with an electronic medical record system, the method comprising, under control of one or more processors of a computing device associated with a patient:
receiving, from a computing system associated with a testing provider or a healthcare provider, an order identifier corresponding to a prescription specifying a user-administered test and the patient;
obtaining a test result corresponding to the user-administered test; and
sending, to the computing system associated with the testing provider or the healthcare provider, the test result in association with the order identifier.

20. The computer-implemented method of claim 19, further comprising:
receiving an indicator of a test type of the user-administered test;
obtaining one or more instructions associated with the test type; and
displaying the one or more instructions to the patient prior to obtaining the test result.

21. The computer-implemented method of claim 20, wherein the one or more instructions comprise instructions for collecting a sample, instructions for application of a sample to a test device, instructions for determination of adequacy of a sample or a test result, or instructions for imaging a test device for determination of a result by the computing device.

22. The computer-implemented method of claim 19, wherein obtaining the test result corresponding to the user-administered test comprises prompting the patient to input the test result at a user interface of the computing device.

23. The computer-implemented method of claim 19, wherein obtaining the test result corresponding to the user-administered test comprises:
receiving an image of a test device used for the user-administered test; and
automatically determining the test result based on image data of the received image of the test device.

24. The computer-implemented method of claim 23, wherein the test device comprises a lateral flow assay test strip, and wherein automatically determining the test result comprises detecting one or more lines on the lateral flow assay test strip.

25. The computer-implemented method of claim 23, wherein the computing device comprises an image capture device, and wherein receiving the image of the test device comprises:
displaying, to the user, an instruction to capture an image of the test device using the image capture device; and
receiving the image from the image capture device.

26. The computer-implemented method of claim 19, further comprising providing a notification to the patient in response to receiving the order identifier.

27. The computer-implemented method of claim 19, wherein the user-administered test comprises a lateral flow assay and the test result comprises one or more lines on the lateral flow assay.
